(19) United States
(12) Patent Application Publication
(10) Pub. No.: US 2016/0128737 A1
(43) Pub. Date: May 12, 2016

(54) SURGICAL IMPLANT SYSTEM AND METHOD

(71) Applicant: Warsaw Orthopedic, Inc., Warsaw, IN (US)

(72) Inventors: Domagoj Coric, Charlotte, NC (US); Mark Dace, Collierville, TN (US)

(21) Appl. No.: 14/534,904

(22) Filed: Nov. 6, 2014

(51) Int. Cl. A61B 17/70

(52) U.S. Cl. CPC A61B 17/7059

(57) ABSTRACT

A method for treating vertebrae includes the steps of: providing a first penetrating element and a second penetrating element; disposing a first penetrating element with a first vertebral body; disposing a second penetrating element with a second vertebral body; removing the first penetrating element from the first vertebral body; providing a spinal implant including at least a first opening and a second opening; disposing the spinal implant adjacent the vertebral bodies such that second opening is aligned with the second penetrating element; providing a fastener; aligning the fastener with the first opening; and attaching the fastener with the first vertebral body. Systems and devices are disclosed.

### TECHNICAL FIELD

The present disclosure generally relates to medical devices for the treatment of musculoskeletal disorders, and more particularly to a surgical system for fastening implants to tissue at a surgical site and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, discectomy, laminectomy, correction and implantable prosthetics. As part of these surgical treatments, implants such as bone fasteners, plates, connectors and vertebral rods are often used to provide stability to a treated region. These implants can redirect stresses away from a damaged or defective region while healing takes place to restore proper alignment and generally support the vertebral members. For example, rods and plates may be attached via the fasteners to the exterior of one or more vertebral members. This disclosure describes an improvement over these prior art technologies.

### SUMMARY

In one embodiment, a method of treating vertebrae is provided. The method comprises the steps of: providing a first penetrating element and a second penetrating element; disposing a first penetrating element with a first vertebral body; disposing a second penetrating element with a second vertebral body; removing the first penetrating element from the first vertebral body; providing a spinal implant including at least a first opening and a second opening; disposing the spinal implant adjacent the vertebral bodies such that second opening is aligned with the second penetrating element; providing a fastener; aligning the fastener with the first opening; and attaching the fastener with the first vertebral body. In some embodiments, systems and devices are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a system in accordance with the principles of the present disclosure;

FIG. 2 is a plan view of components of one embodiment of a system in accordance with the principles of the present disclosure disposed with vertebrae;

FIG. 3 is a plan view of components of one embodiment of a system in accordance with the principles of the present disclosure disposed with vertebrae;

FIG. 4 is a plan view of components of one embodiment of a system in accordance with the principles of the present disclosure disposed with vertebrae;

FIG. 5 is a plan view of components of one embodiment of a system in accordance with the principles of the present disclosure disposed with vertebrae;

FIG. 6 is a side view of components of one embodiment of a system in accordance with the principles of the present disclosure disposed with vertebrae;

FIG. 7 is a plan view of components of one embodiment of a system in accordance with the principles of the present disclosure disposed with vertebrae;

FIG. 8 is a plan view of components of one embodiment of a system in accordance with the principles of the present disclosure disposed with vertebrae;

FIG. 9 is a plan view of components of one embodiment of a system in accordance with the principles of the present disclosure disposed with vertebrae;

FIG. 10 is a plan view of components of one embodiment of a system in accordance with the principles of the present disclosure disposed with vertebrae; and

FIG. 11 is a plan view of components of one embodiment of a system in accordance with the principles of the present disclosure disposed with vertebrae.

### DETAILED DESCRIPTION

The exemplary embodiments of a surgical implant system are discussed in terms of medical devices for the treatment of musculoskeletal disorders and more particularly, in terms of a surgical implant system for delivering and/or fastening implants with a surgical site and a method for treating a spine.

In some embodiments, the present surgical implant system and method are employed for installing a spinal implant, such as, for example, an anterior cervical plate by using a hole employed for a penetrating element, such as, for example, a distractor pin and using that hole as a screw hole for a bone screw to attach the plate to vertebrae. In some embodiments, the present surgical implant system and method are employed for inserting one screw per vertebral body plate. In some embodiments, the present surgical implant system and method employ distraction pins and/or holes as guides. In some embodiments, the present surgical implant system and method reduce the number of steps in a surgical technique, as described herein.

In some embodiments, the present surgical implant system and method are employed with a surgical technique including delivery of one or more system components over one or more distraction pins. In some embodiments, the distraction pins can be used to initiate a screw hole and provide distraction while discectomy is performed. In some embodiments, the distraction pins are designed to facilitate plate placement over the distraction pins. In some embodiments, the plate can be introduced over the distraction pins, and the pins can be removed via a pin driver.

In some embodiments, the present surgical implant system and method are employed with a surgical technique including a step of disc space distraction such that the distraction pins are placed in a midline of vertebrae, which may be selectively aligned for a final bone screw placement. In some embodiments, the method includes the step of determining an approximate final bone screw trajectory with initial pin placement. In some embodiments, the method includes the step of performing discectomy/decompression under distraction. In some embodiments, the method includes the step of placing an interbody spacer with the vertebrae. In some embodiments, the method includes the step of delivering a plate such that a lower distraction pin is removed and the plate is placed over the remaining distraction pin. In some embodiments, the method includes the step of securing the plate with a bone screw in the lower hole where the lower distraction pin was removed. In some embodiments, the method includes the step of removing the remaining distraction pin and placing a final bone screw in the upper hole where the upper distraction pin was removed. In some embodiments, the method includes the step of performing final tightening of the screws and rotating lock cap(s) to a secured plate position.

In one embodiment, the surgical implant system includes a spinal plate and a method of insertion thereof. In one embodiment, the surgical implant system utilizes a method including the step of distracting a disc space to access a posterior and/or posterolateral disc space to perform a step of decompression of a spine. In one embodiment, distraction pins are inserted in a coronal midline of the vertebral bodies. In one embodiment, a distraction pin placement tool selectively places distraction pins away from endplates so that the plate position on the vertebral body accommodates bone screws in the distraction pin holes. In some embodiments, AP fluoroscopy may be used to verify a midline alignment before pin placement.

In one embodiment, a pin driver and the distraction pin placement tool are utilized to insert a distraction pin in a coronal midline of a cranial vertebral body and parallel to a caudal vertebral end plate. In one embodiment, a second distraction pin is inserted on the coronal midline of the caudal vertebral body and parallel to the first distraction pin. In some embodiments, the system may include multiple distraction pin lengths.

In some embodiments, the present surgical implant system and method are employed with a surgical technique including a step of decompressing vertebrae. In some embodiments, the method includes the step of resecting the proximal uncovertebral joints along with any osteophytes. In some embodiments, a posterior longitudinal ligament may also be removed, and the nerve roots decompressed. In some embodiments, the method includes the step of inserting an interbody spacer, such as, for example, autograft, allograft or other interbody fusion device in the cervical spine. In some embodiments, the method includes the step of dissecting soft-tissue and removing anterior osteophytes to provide a bone-plate interface.

In some embodiments, once the graft is in position, the method includes the step of removing the distractor and leaving the distraction pins in the bone. In one embodiment, the method includes the step of removing the caudal distraction pin and a spinal plate is disposed over the remaining distraction pin. In one embodiment, a screw is inserted into the caudal bone screw hole. In one embodiment, a rostral distraction pin is removed and inserted with the bone screw. In one embodiment the method includes the step of packing bone into an interspace around the plate and the interbody spacer.

In some embodiments, the spinal plate includes a locking cap mechanism configured to provide visual and tactile confirmation that the lock is rotated over the head of the screws. In some embodiments, the locking cap has a positive stop that prevents the cap from rotating more than 90 degrees clockwise. In some embodiments, the locking cap is prevented from turning counterclockwise to prevent damage to the locking mechanism. In one embodiment, an instrument, such as, for example, a screwdriver is inserted into a head of the locking cap and rotated 90 degrees until the cap covers both screw heads and a positive stop is felt.

In some embodiments, the system includes one or more anterior cervical plates, which may include two or three holes. In some embodiments, the plates may be provided in lengths ranging from 18 millimeters (mm) to 30 mm for one level procedures and 28 mm to 50 mm for two level procedures. In one embodiment, a plate holder is utilized to select the appropriate plate size and place it on a vertebral column. In some embodiments, lateral fluoroscopy may be used to determine a selected plate length and screw trajectories.

In some embodiments, the anterior cervical plate is provided with a pre-machined lordotic curve. In some embodiments, the anterior cervical plate may be contoured to increase or decrease lordotic curvature by using a plate bender. In some embodiments, a gradual bend can be made avoiding bending of the plate in the region of the lock to avoid weakening the plate and/or prevent proper actuation of the locking mechanism. In some embodiments, bone screws may comprise self-drilling and/or self-tapping configurations. In some embodiments, the screws are color-coded to indicate diameter.

In some embodiments, the present surgical implant system and method are employed with a surgical technique including the step of holding the plate in position, such as, for example, via use of temporary fixation pins, a plate holder and/or a drill, tap, screw (DTS) guide. In some embodiments, after the plate is positioned and aligned with the midline of the anterior cervical spine, an awl may be used to mark the entry points for the screws. In some embodiments, the method includes the step of inserting a tri-flat end of the awl shaft into a universal handle. In some embodiments, the method includes the step of securely seating a guide in an aperture of the bone screw hole on the plate and inserting the awl into the guide. In some embodiments, the method includes the step of applying downward pressure on the awl to puncture a cortex of the bone such that the awl will protrude into the bone a maximum of 10 mm.

In some embodiments, the surgical implant system includes a DTS guide that allows the awl, drill bits, tap and screws to pass through the guide. In some embodiments, the plate includes at least one lateral edge to allow engagement with the DTS guide. In some embodiments, the DTS guide is attached to the plate by engaging distal ends thereof over the lateral edges of the plate at the perimeter of a screw hole. In some embodiments, the DTS guide may be used to position the plate onto the vertebral body. In some embodiments, the DTS guide includes a variable positioning angle of 18 degrees to 0 degrees.

In some embodiments, the surgical implant system of the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the surgical implant system of the present disclosure may be employed with other osteal and bone related applications, including those associated with diagnostics and therapeutics. In some embodiments, the disclosed surgical implant system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The surgical implant system of the present disclosure may also be alternatively employed with procedures for treating the lumbar, cervical, thoracic, sacral and pelvic regions of a spinal column. The surgical implant system of the present disclosure may also be used on animals, bone models and other non-living substrates, such as, for example, in training, testing and demonstration.

The surgical implant system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

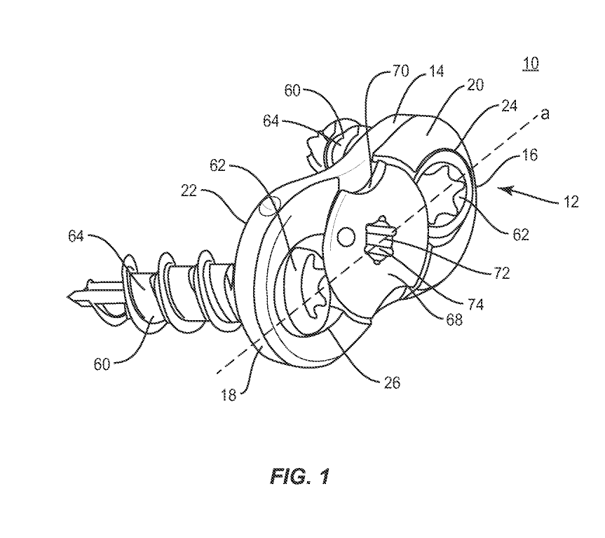

The following discussion includes a description of a surgical implant system, related components and methods of employing the surgical implant system in accordance with the principles of the present disclosure. Alternate embodiments are also disclosed. Reference is made in detail to the exemplary embodiments of the present disclosure, which are illustrated in the accompanying figures. Turning to FIG. 1, there are illustrated components of a surgical implant system 10.

The components of surgical implant system 10 can be fabricated from biologically acceptable materials suitable for medical applications, including metals, synthetic polymers, ceramics and bone material and/or their composites. For example, the components of surgical implant system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of surgical implant system 10 may have material composites, including the above materials, to achieve various desired characteristics such as strength, rigidity, elasticity, compliance, biomechanical performance, durability and radiolucency or imaging preference. The components of surgical implant system 10, individually or collectively, may also be fabricated from a heterogeneous material such as a combination of two or more of the above-described materials. The components of surgical implant system 10 may be monolithically formed, integrally connected or include fastening elements and/or instruments, as described herein.

Surgical implant system 10 is employed, for example, with an open or mini-open, minimal access and/or minimally invasive including percutaneous surgical technique to deliver and fasten an implant, such as, for example, an anterior cervical plate at a surgical site within a body of a patient, for example, a section of a spine. In one embodiment, the components of surgical implant system 10 are configured to temporarily fix an implantable plate with a provisional fixation element, such as, for example, those penetrating elements and/or bone fasteners described herein, to selectively position and orient the plate relative to tissue for a surgical treatment to treat various spine pathologies, such as those described herein. In one embodiment, upon selected positioning and orientation of the plate, implantable bone fasteners of surgical implant system 10 are employed to fix the plate with tissue and the provisional fixation element is removed from the tissue. In some embodiments, the provisional fixation element can be removed from tissue before or after fixation of the bone fasteners. In one embodiment, a bone fastener includes components implanted with tissue and remaining after a surgical procedure is completed and an incision is dosed. In one embodiment, a bone fastener includes components implanted with tissue and remaining after a first and/or initial surgical procedure is completed and an incision is dosed, and the component is removable in a second, subsequent and/or separate surgical procedure.

Surgical implant system 10 includes a spinal implant, such as, for example, an anterior cervical plate 12. In some embodiments, plate 12 is configured for connecting two vertebral bodies, each vertebral body receiving one bone fastener through each opening. Plate 12 has a substantially oblong shape and a continuous lordotic curve along its length to accommodate the curvature of the spinal column. In some embodiments, plate 12 is variously shaped, such as, for example, rectangular, oval, triangular, polygonal, irregular, uniform, non-uniform, variable and/or tapered. In some embodiments, plate 12 is configured for connecting three or more vertebral bodies. In some embodiments, each vertebral body receives at least one bone fastener through each opening thereof.

Plate 12 is elongated such that when plate 12 is disposed with vertebrae, plate 12 extends between a midline axis M1 of a vertebral body V1 and a vertebral body V2, as shown in FIG, 3. Plate 12 defines a longitudinal axis X1 disposed in alignment with midline axis M1.

Plate 12 includes a wall 14 extending between an end 16 and an end 18. Wall 14 defines a longitudinal axis a. Wall 14 has a surface 20 configured for orientation in an anterior direction of a body and a surface 22 configured for orientation in a posterior direction to engage an anterior portion of vertebrae V. In some embodiments, surface 20 and/or surface 22 may have various surface configurations, such as, for example, rough, threaded, arcuate, undulating, porous, semi-porous, dimpled, polished and/or textured.

Plate 12 defines a series of openings, such as, for example, openings 24, 26 extending through surfaces 20, 22. Openings 24, 26 are configured for disposal of implantable bone fasteners, such as, for example, bone screws 60 for attaching plate 12 with tissue. Openings 24, 26 are centrally disposed along axis a for alignment with midline axis M1. Openings 24, 26 are configured for slidable disposal of a penetrating element, such as, for example, a distraction pin 80 for temporary fixation in a selected orientation with vertebrae V. as described herein. In some embodiments, plate 12 includes a width of 9-10.5 mm and a maximum thickness of 2.2 mm.

Bone screw 60 comprises a head 62 and an elongated shaft 64 configured for penetrating tissue. In some embodiments, surgical implant system 10 may include one or a plurality of bone fasteners and/or penetrating dements. The shaft of bone screw 60 has a cylindrical cross section configuration and includes an outer surface having an external threaded form (not shown). In some embodiments, the thread form may include a single thread turn or a plurality of discrete threads. In some embodiments, other engaging structures may be located on the shaft of bone screw 60, such as, for example, a nail configuration, barbs, expanding elements, raised elements and/or spikes to facilitate engagement of shaft 64 with tissue, such as, for example, vertebrae. In some embodiments, one or more bone screws 60 include a length of 10 through 24 mm. In some embodiments, one or more bone screws 60 include a major diameter of 4 mm, a minor diameter of 3 mm and a pitch of 2.3. In some embodiments, one or more bone screws 60 include a major diameter of 4.5 mm, a minor diameter of 3 mm and a pitch of 2.3. In some embodiments, one or more bone screws 60 include a major diameter of 5 mm, a minor diameter of 3 mm and a pitch of 2.3.

In some embodiments, all or only a portion of the shaft of bone screw 60 may have alternate cross section configurations, such as, for example, oval, oblong, triangular, square, polygonal, irregular, uniform, non-uniform, offset, staggered, undulating, arcuate, variable and/or tapered. In some embodiments, the outer surface of the shaft of bone screw 60 may include one or a plurality of openings. In some embodiments, all or only a portion of the outer surface of the shaft of bone screw 60 may have alternate surface configurations to enhance fixation with tissue such as, for example, rough, arcuate, undulating, mesh, porous, semi-porous, dimpled and/or textured according to the requirements of a particular application. In some embodiments, all or only a portion of the shaft of bone screw 60 may be disposed at alternate orientations, relative to its longitudinal axis, such as, for example, transverse, perpendicular and/or other angular orientations such as acute or obtuse, co-axial and/or may be offset or staggered. In some embodiments, all or only a portion of the shaft of bone screw 60 may be cannulated.

Plate 12 includes retaining elements 68 disposed between openings 24, 26 so as to resist and/or prevent inadvertent back out of bone screws 60 after bone screws 60 have been fully inserted into openings 24, 26. Plate 12 includes a recess 70 in surface 20 dimensioned to correspond to retaining element 68. Retaining element 68 is configured to engage recess 70 such that retaining element 68 is rotatable between a locked orientation and a non-locking orientation. In the locked orientation, retaining element 68 resists and/or prevents backout of bone screws 60 from openings 24, 26. In the non-locking orientation, bone screws 60 are axially translatable through openings 24, 26. In one embodiment, plate 12 includes one retaining element 68. In some embodiments, plate 12 includes two or more retaining elements 68 each being disposed between each pair of openings 24, 26 in plate 12.

Retaining element 68 includes an inner surface 72 that defines a centrally disposed aperture 74 extending through surfaces 20, 22 of plate 12. Aperture 74 is configured for disposal of an instrument, such as, for example, a driver 96. Inner surface 72 has a hexagonal configuration configured to engage a correspondingly shaped portion of driver 96 for rotating retaining element 68 with respect to recess 70 between the locked and nonlocking orientations. In some embodiments, inner surface 72 may be variously configured to engage, such as, for example, a Phillips head, slotted head, hex socket head, hexagon external head, frearson head, square socket, square slotted combination head, spanner drilled tamper proof head and combinations thereof.

Distraction pins 80a, 80b include a shaft 82a, 82b configured to penetrate tissue. Shafts 82a, 82b are configured to penetrate tissue including bone for distracting a spine and/or vertebrae, and/or may provide temporary and/or provisional fixation of components of spinal implant system 10 with tissue. In some embodiments, distraction pins 80a, 80b are configured for insertion in a coronal midline of vertebral bodies. A distraction pin placement tool (not shown) is utilized to position distraction pins 80a, 80b a distance from endplates of vertebrae. Pins 80a, 80b form cavities 84a, 84b so that plate 12 can be positioned on one or more vertebral bodies to accommodate bone screws 60 for attachment of plate 12 with vertebrae, as described herein. In some embodiments, one or more pins 80 include a length of 12 mm, a major diameter of 2.72 mm, a minor diameter of 2.03 mm and a pitch of 0.98. In some embodiments, one or more pins 80 include a length of 14 mm, a major diameter of 2.72 mm, a minor diameter of 2.03 mm and a pitch of 0.98. In some embodiments, one or more pins 80 may include a threaded surface, as described herein, and/or include an even or smooth surface.

In assembly, operation and use, surgical implant system 10, similar to the systems and methods described herein, is employed with a surgical procedure for treatment of a spinal disorder affecting a section of a spine of a patient, as discussed herein. For example, surgical implant system 10 can be used with a surgical procedure for treatment of a condition or injury of an affected section of the spine including vertebrae. In some embodiments, one or all of the components of surgical implant system 10 can be delivered or implanted as a pre-assembled device or can be assembled in situ. Surgical implant system 10 may be completely or partially revised, removed or replaced.

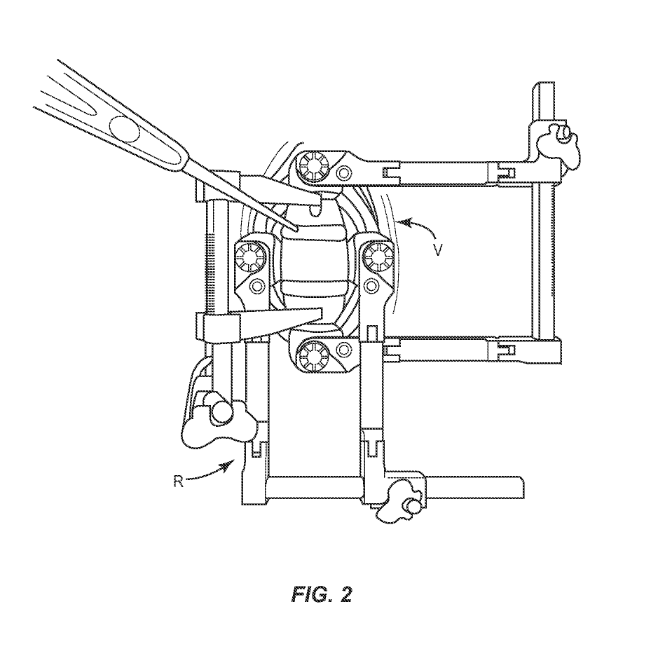

For example, surgical implant system 10 can be employed with a surgical treatment of an applicable condition or injury of an affected section of a spinal column and adjacent areas within a body, such as, for example, cervical vertebrae V, as shown in FIGS. 2-11. In some embodiments, surgical implant system 10 may be employed with one or a plurality of vertebra. To treat a selected section of vertebrae V, a medical practitioner obtains access to a surgical site including vertebrae V in any appropriate manner. In some embodiments, surgical implant system 10 can be used in any existing surgical method or technique including open surgery, mini-open surgery, minimally invasive surgery and percutaneous surgical implantation, whereby vertebrae V are accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of surgical implant system 10, as shown in FIG. 2. Tissue is spaced with a retractor R. A preparation instrument (not shown) can be employed to prepare tissue surfaces of vertebrae V, as well as for aspiration and irrigation of a surgical region.

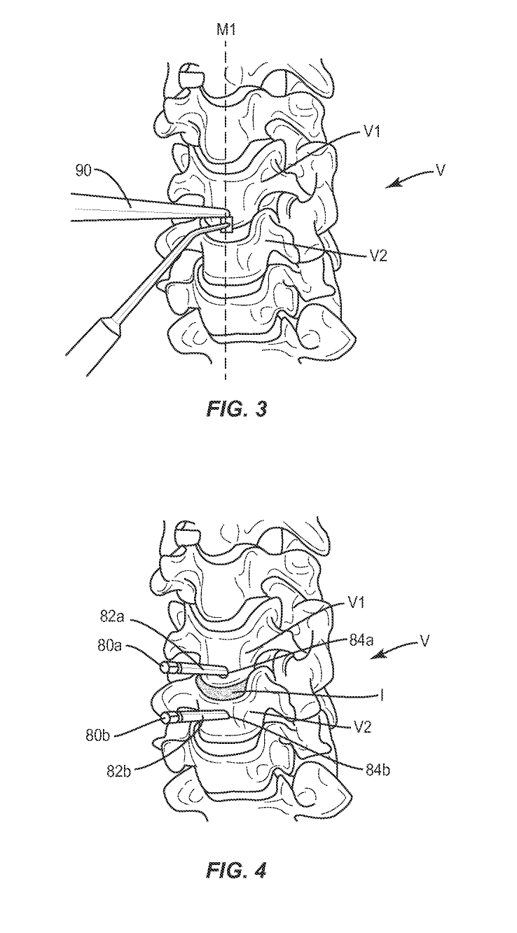

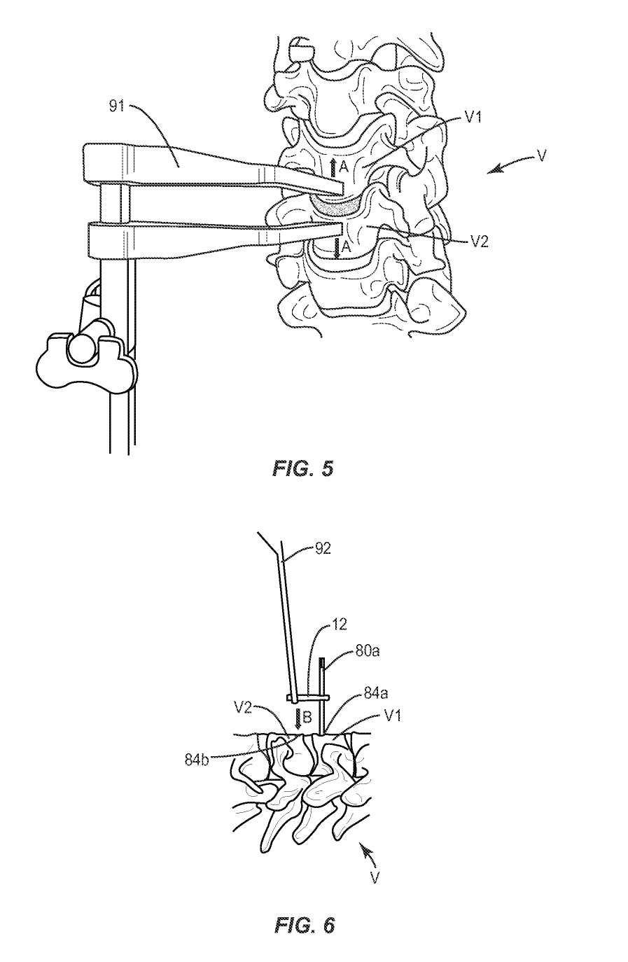

A distraction pin placement tool 90, as shown in FIGS. 3 and 4, is employed to insert a distraction pin 80a along midline axis M1 in a coronal plane of a cranial vertebral body V1 and parallel to a vertebral end plate of a caudal vertebral body V2. A second distraction pin 80b is inserted along midline axis M1 in a coronal plane of caudal vertebral body V2 and parallel to distraction pin 80a. Disposal of pins 80a, 80b create holes 84a, 84b in vertebrae V1, V2, respectively. In some embodiments, tool 90 is configured to place pins 80a, 80b a selective distance from the endplates of vertebrae V1, V2 such that position of plate 12 will be sufficient to accommodate bone screws 60 in holes 84a, 84b. In some embodiments, AP fluoroscopy may be used to verify midline axis M1. In some embodiments, a distraction tool 91, as shown in FIG. 5, is employed to distract, in the directions shown by arrows A, an intervertebral disc space I to access the posterior and/or posterolateral disc space for decompression of the spine.

Distraction pins 80a, 80b are maintained in vertebrae V1, V2, as shown in FIG. 4. In some embodiments, a discectomy is performed. In some embodiments, the proximal uncovertebral joints may be resected along with any osteophytes. In some embodiments, the posterior longitudinal ligament may be removed, and the nerve roots decompressed. In some embodiments, an interbody spacer, such as, for example, autograft, allograft or any interbody fusion device is implanted with space I.

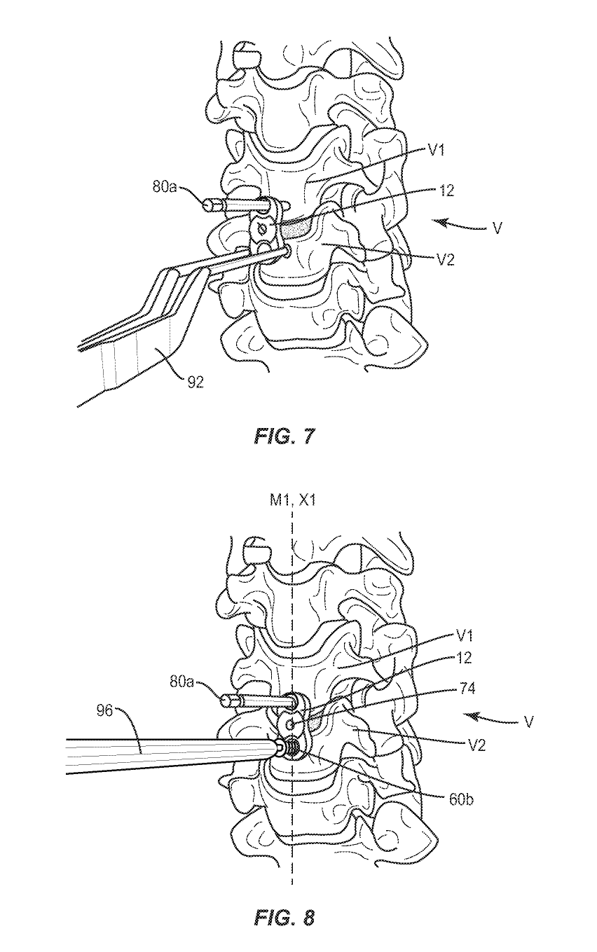

Distraction pin 80b is removed from caudal vertebral body V2, as shown in FIGS. 6 and 7. Plate 12 is delivered to the surgical site adjacent vertebrae V with an insertion tool 92 and over distraction pin 80a, as shown by arrow B in FIG. 6. Plate 12 is disposed in a selected position and orientation relative to vertebrae V such that surface 22 of plate 12 is disposed with tissue and openings 24, 26 are disposed in alignment with midline axis M1 of vertebrae V1, V2. Opening 24 is translated over distraction pin 80a and opening 26 is oriented such that opening 26 is in alignment with hole 84b. Distraction pin 80a temporarily fixes plate 12 with vertebrae V in a selected position and orientation such that axis a of plate 12 is aligned with midline axis M1, as shown in FIG. 8. In some embodiments, a bone screw 60 diameter, length and/or pitch is selected, as described herein.

A bone screw 60b is delivered to the surgical site and disposed in hole 84b formed in caudal vertebral body V2. The components of surgical implant system 10 including a driver 96 are manipulable to drive, torque, insert or otherwise connect bone screw 60b with vertebral body V2 for fastening plate 12 with vertebrae V.

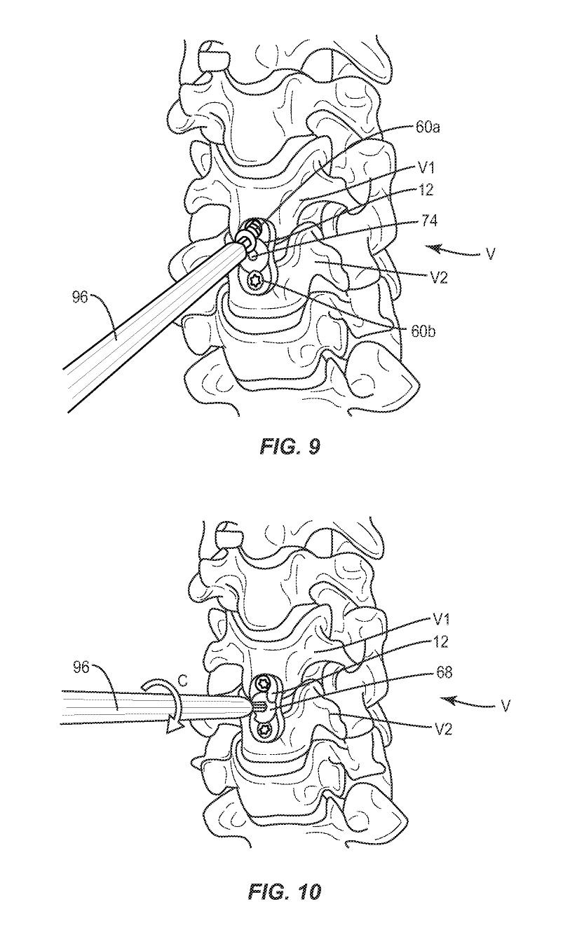

Distraction pin 80a is removed from cranial vertebral body V1, as shown in FIG. 9. A bone screw 60a is delivered to the surgical site and disposed in hole 84a formed in cranial vertebral body V1. Driver 96 is manipulable to drive, torque, insert or otherwise connect bone screw 60a with vertebral body V1 for fastening plate 12 with vertebrae V. Bone screws 60a, 60b are fully inserted and implanted through openings 24, 26 of plate 12 with tissue to implant plate 12 in the selected position and orientation.

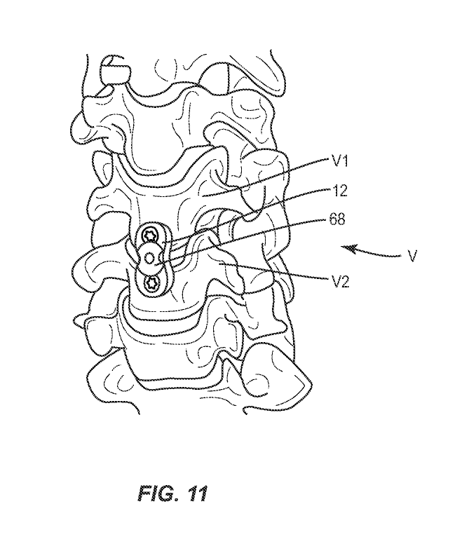

Driver 96 is positioned within aperture 74 to rotate, as shown by arrow C in FIG. 10, retaining element 68 from the non-locking orientation to the locked orientation, as shown in FIG. 11, such that retaining element 68 partially overlaps bone screws 60 and openings 24, 26 to resist and/or prevent inadvertent back out of bone screws 60 from plate 12 and/or tissue. In some embodiments, the surgical treatment includes selecting one or more bone screws 60 having a larger shaft diameter and/or length than distraction pin 80 to resist and/or prevent loosening and/or backout of bone screw 60 from holes 84. In some embodiments, the surgical treatment includes selecting one or more bone screws 60 having a larger shaft diameter such that a minor diameter of bone screw 60 is larger than a major diameter of distraction pin 80. In some embodiments, the surgical treatment includes selecting a bone screw having a shaft diameter that is similar to a shaft diameter of a distraction pin such that a pitch of the bone screw is equal to a pitch of threads of a distraction pin. In some embodiments, the surgical treatment includes selecting one or more bone screws having parameters, as described herein, specifically based on one or more parameters, as described herein, of the distraction pin to resist and/or prevent loosening and/or backout of the bone screw from holes 84.

Upon completion of the procedure, the surgical instruments, assemblies and non-implanted components of surgical implant system 10 are removed from the surgical site and the incision is closed. One or more of the components of surgical implant system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of surgical implant system 10.

In some embodiments, surgical implant system 10 may include one or a plurality of plates, connectors, spinal rods and/or bone fasteners for use with a single vertebral level or a plurality of vertebral levels. In some embodiments, the plates, connectors, spinal rods and/or bone fasteners may be engaged with vertebrae in various orientations, such as, for example, series, parallel, offset, staggered and/or alternate vertebral levels. In some embodiments, the bone fasteners may include one or a plurality of multi-axial screws, sagittal angulation screws, pedicle screws, mono-axial screws, uni-planar screws, fixed screws, tissue penetrating screws, conventional screws, expanding screws, anchors, buttons, connectors, clips, snaps, friction fittings, compressive fittings, expanding rivets, staples, nails, adhesives, plates and/or posts.

In one embodiment, surgical implant system 10 includes an agent, which may be disposed, packed, coated or layered within, on or about the components and/or surfaces of surgical implant system 10. In some embodiments, the agent may include bone growth promoting material, such as, for example, bone graft to enhance fixation of the bone fasteners with vertebrae. In some embodiments, the agent may include one or a plurality of therapeutic agents and/or pharmacological agents for release, including sustained release, to treat, for example, pain, inflammation and degeneration.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A method for treating vertebrae, the method comprising the steps of:
providing a first penetrating element and a second penetrating element;
disposing a first penetrating element with a first vertebral body;
disposing a second penetrating element with a second vertebral body;
removing the first penetrating element from the first vertebral body;
providing a spinal implant including at least a first opening and a second opening;
disposing the spinal implant adjacent the vertebral bodies such that second opening is aligned with the second penetrating element;
providing a fastener;
aligning the fastener with the first opening; and
attaching the fastener with the first vertebral body.

2. A method as recited in claim 1, wherein the penetrating dements include distractor pins.

3. A method as recited in claim 1, further comprising the step of selecting elements of the fasteners comprising diameter, length and/or pitch.

4. A method as recited in claim 1, wherein the spinal implant comprises a plate and further comprising the step of selecting elements of the plate comprising size and/or configuration.

5. A method as recited in claim 1, wherein the spinal implant comprises an anterior cervical plate.

6. A method as recited in claim 1, further comprising the step of aligning the penetrating elements along a coronal midline plane.

7. A method as recited in claim 1, wherein the first vertebral body includes a cranial vertebral body and the second vertebral body includes a caudal vertebral body and further comprising the step of aligning the penetrating elements along a coronal midline plane.

8. A method as recited in claim 1, wherein the penetrating elements are disposed in parallel distraction planes.

9. A method as recited in claim 1, further comprising the step of distracting the first vertebral body relative to the second vertebral body.

10. A method as recited in claim 1, further comprising the step of performing a discectomy and/or a decompression.

11. A method as recited in claim 1, further comprising the step of implanting an interbody implant between the first vertebral body and the second vertebral body.

12. A method as recited in claim 1, wherein the step of delivering the spinal implant includes translating the spinal implant over the second penetrating element.

13. A method as recited in claim 1, further comprising the step of removing the second penetrating element.

14. A method as recited in claim 1, further comprising the step of providing a second fastener for alignment with the second opening.

15. A method as recited in claim 14, further comprising the step of attaching the second fastener with the second vertebral body.

16. A method as recited in claim 15, further comprising the step of locking the first fastener and the second fastener.

17. A method for treating vertebrae, the method comprising the steps of:
providing a first distractor pin and a second distractor pin;
disposing the first pin with a first vertebra of a subject body;
disposing the second pin with a second vertebra such that the pins are substantially aligned with a coronal midline plane of the subject body;
removing the first pin from the first vertebra;
providing a spinal implant including at least a first opening and a second opening;
delivering the spinal implant adjacent the vertebrae such that second opening is aligned with the second pin;
providing a first fastener;
aligning the first fastener with the first opening;
attaching the first fastener with the first vertebral body;
removing the second pin from the second vertebra;
providing a second fastener;
aligning the second fastener with the second opening;
attaching the second fastener with the second vertebral body such that the openings are substantially aligned with the coronal midline plane.

18. A method as recited in claim 17, further comprising the step of selecting screw diameter, length and/or pitch of the fasteners.

19. A method as recited in claim 17, wherein the spinal implant comprises a plate and further comprising the step of selecting elements of the plate comprising size and/or configuration.

20. A method for treating vertebrae, the method comprising the steps of:
providing a first penetrating element and a second penetrating element;
disposing a first penetrating element with a first vertebral body;
disposing a second penetrating element with a second vertebral body;
removing the first penetrating element from the first vertebral body;
providing an anterior cervical plate including at least a first opening and a second opening;
delivering the spinal implant along an anterior surgical pathway and adjacent the vertebral bodies such that second opening is aligned with the second penetrating element;
providing a fastener;
aligning the fastener with the first opening;
attaching the fastener with the first vertebral body such that the openings are disposed in a coronal midline orientation.
